(19) United States
(12) Patent Application Publication
(10) Pub. No.: US 2011/0117042 A1
(43) Pub. Date: May 19, 2011

(54) POLYURETHANE DISPERSIONS FOR HAIR DYE

(75) Inventors: Sophie Viala, Koln (DE); Sebastian Dörr, Dusseldorf (DE); Steffen Hofacker, Odenthal (DE)

(73) Assignee: Bayer MaterialScience, Leverkusen (DE)

(21) Appl. No.: 13/054,555

(22) PCT Filed: Jul. 4, 2009
(86) PCT No.: PCT/EP09/04834

§ 371 Date: Jan. 17, 2011

(30) Foreign Application Priority Data
Jul. 18, 2008 (EP) 08012988.5

(51) Int. Cl. A61K 8/87; A61Q 5/10; A61Q 5/06; B65D 83/14

(52) U.S. Cl. 424/70.6; 8/406; 222/635

(57) ABSTRACT

The present invention relates to a cosmetic composition for coloring a keratin-containing material, which includes one or more aqueous polyurethane dispersions

The present invention relates to hair colorants comprising special polyurethanes and to the use of said polyurethanes for preparing hair colorant compositions.

A variety of products is supplied for colouring hair: temporary direct, semipermanent and permanent colorants. These hair colorants differ in their colouring mechanisms and their consumer properties.

The use of direct temporary hair colorants achieves only a certain nuance of an existing hair colour. The dyes used (for example food or vegetable dyes) are deposited on the surface of the hair. Consequently, these hair colorants are easily removed with shampoo.

In the case of the semipermanent colorant, in most cases nonionic or cationic substances with a low molecular weight are used which colour the hair by diffusing in without altering the chemical structure. In contrast to direct temporary hair colorants, they are less easy to remove. They withstand 5 to 6 hair washes.

In the case of permanents hair colorants, also called oxidation dyes, the dyes are produced and bonded directly on and in the hair through chemical reactions. The oxidation dyes are colourless precursors. Through hydrogen peroxide as oxidizing agent, oxidation reactions and coupling reactions and/or condensations take place. This results in the coloured compounds of varying degree of polymerization.

The aforementioned hair colorants have their advantages and disadvantages. Since the colouring of hair in the case of the oxidation dyes is achieved through the use of strong oxidizing agents such as, for example, hydrogen peroxide, the hair is greatly stressed. This results in damage to the hair. On the other hand, the direct temporary hair colorants are gentle to the hair. However, the results of colouring damaged hair are anyway often not satisfactory. Moreover, these hair colorants are less resistant to washing. For this reason, many products contain a combination of different dye types in order to achieve the desired properties such as, for example, natural colour shades.

EP 1695688 describes anhydrous formulations comprising a film-forming polymer and at least one direct cationic or nonionic hair colorant. However, aqueous polyurethane dispersions are not mentioned.

WO 99/36047 discloses compositions of 2 parts of oxidation hair colorant and nonionic polyether polyurethanes. The use or the advantages of aqueous polyurethane dispersions are not described.

Surprisingly, it has now been found that the use of aqueous polyurethane dispersions can bring about an improved, long-lasting colouring result.

The present invention therefore provides cosmetic compositions for colouring keratin-containing materials, preferably hair, comprising one or more aqueous polyurethane dispersions, and also cosmetic compositions for colouring the hair in the preparation of which one or more aqueous polyurethane dispersions are used.

Polyurethanes within the context of the invention are polymeric compounds which have at least two urethane-group-containing repeat units:

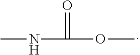

Also included according to the invention are those polyurethanes which, as a result of the preparation, also have urea-group-containing repeat units:

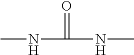

as are formed in particular during the chain extension of isocyanate-terminated prepolymers with polyamines.

The use of these polyurethanes as aqueous dispersions surprisingly leads to an improvement in the colouring and to a long-lasting duration of the colouring effect.

In the cosmetic compositions according to the invention, the polyurethane is generally present in dispersed, i.e. non-dissolved, form. The cosmetic compositions according to the invention are in particular water-containing, i.e. aqueous, compositions in which the polyurethane is present in dispersed form. Besides other liquid media which may be present if desired, such as, for example, solvents, water generally forms the main constituent (>50% by weight) based on the liquid dispersion media in the cosmetic compositions according to the invention, optionally also the only liquid dispersion medium.

The polyurethanes present in the cosmetic compositions according to the invention preferably comprise a sequence which is selected from the group which consists of: polyester, polyether, polycarbonate and polyether-polycarbonate sequences. According to the invention, this means that the polyurethanes contain ester-, ether- and/or carbonate-containing repeat units.

The polyurethanes according to the invention are preferably essentially linear molecules, but may also be branched, which is less preferred.

The number-average molecular weight of the polyurethanes used preferably according to the invention is typically from 1000 to 100 000, preferably from 5000 to 50 000 g/mol.

The polyurethanes present in the cosmetic compositions according to the invention are added to said compositions in particular as aqueous dispersions. Such aqueous polyurethane dispersions are obtainable in particular by a process which involves the reaction of the following components:
A1) one or more organic polyisocyanates,
A2) one or more polymeric polyols, preferably with number-average molecular weights of from 400 to 8000 g/mol (here and for the molecular weight data below, determined by gel permeation chromatography against polystyrene standard in tetrahydrofuran at 23° C.), more preferably 400 to 6000 g/mol and particularly preferably from 600 to 3000 g/mol, and OH functionalities of from preferably 1.5 to 6, more preferably 1.8 to 3, particularly preferably from 1.9 to 2.1,
A3) optionally one more hydroxy-functional compounds, preferably with molecular weights of from 32 to less than 400 g/mol,
A4) optionally one or more isocyanate-reactive hydrophilizing agents, which are preferably selected from anionic, potentially anionic and/or nonionic hydrophilizing agents, and
B1) optionally one or more amino-functional compounds, preferably with molecular weights of from 32 to 400 g/mol.

The aqueous polyurethane dispersions present in the cosmetic compositions according to the invention are more preferably obtainable by a process which involves the following process steps:

process for the preparation of an isocyanate-functional prepolymer which involves the reaction of the following components:
A1) one or more organic polyisocyanates,
A2) polymeric polyols, preferably with number-average molecular weights of from 400 to 8000 g/mol (here and for the molecular weight data below, determined by gel permeation chromatography against polystyrene standard in tetrahydrofuran at 23° C.), more preferably 400 to 6000 g/mol and particularly preferably from 600 to 3000 g/mol, and OH—functionalities of from preferably 1.5 to 6, more preferably 1.8 to 3, particularly preferably from 1.9 to 2.1,
A3) optionally one or more hydroxy-functional compounds, preferably with molecular weights of from 32 to less than 400 g/mol,
A4) optionally one or more isocyanate-reactive hydrophilizing agents, which are preferably selected from anionic, potentially anionic and/or nonionic hydrophilizing agents,
   (where the stoichiometry is selected such that the resulting prepolymers are isocyanate-termated), and
   process for the preparation of the polyurethane, which involves:
   the reaction of the isocyanate-functional prepolymer obtained in step A) with one or more of the components which are selected from the group which consists of:
B1) one or more amino-functional compounds, preferably with molecular weights of from 32 to 400 g/mol and
B2) one or more isocyanate-reactive, preferably amino-functional hydrophilizing agents, which are preferably selected from anionic and potentially anionic hydrophilizing agents.

In step B), a chain extension of the isocyanate-functional prepolymer obtained in step A) generally takes place.

In particular, the amino-functional compound B) comprises at least one amino-functional compound B1) which has no ionic and/or ionogenic groups. This is preferably a diamine which has no ionic and/or ionogenic groups.

Preferably, in step B), both component B1) and also component B2) are used.

The dispersion of the polyurethanes in water can take place before, during or after step B), where any potentially ionic groups present are converted to the ionic form by partial or complete reaction with a neutralizing agent.

In order to achieve the anionic hydrolyphilization preferred according to the invention, the anionic or potentially anionic components A4) and/or B2) must be used as hydrophilizing agents, which is preferably the case. These preferably have at least one group which is reactive towards NCO groups, such as preferably an amino, hydroxy or thiol group, and moreover at least one anionic group or group that can be converted to an anionic group, such as —COO⁻, —SO₃⁻ or —PO₃²⁻ and/or their completely or partially protonated acid forms. Further details of this are given below. Particular preference is given to sulphonate groups as anionic groups. The use of sulphonate groups as anionic groups in the polyurethanes used according to the invention leads to a greater resistance towards electrolytes, such as salts, the establishment of a neutral pH of the dispersions is simplified, and the dispersions remain stable over a broader pH, such as a pH of from about 5 to 8. The use of the sulphonate-group-carrying polyurethanes also increases the stability of the cosmetic compositions according to the invention.

The preferred aqueous, anionic polyurethane dispersions have a low degree of hydrophilic anionic groups, preferably from 0.1 to 15 milliequivalents per 100 g of solid resin. A relatively high degree of anionic groups is disadvantageous because, in some cases, the drying of the resulting film from the cosmetic composition could be slowed.

In order to achieve good sedimentation stability, the number-average particle size of the special polyurethane dispersions is preferably less than 750 nm, particularly preferably less than 500 nm and very particularly preferably less than 400 nm, determined by means of laser correlation spectroscopy following dilution with deionized water (instrument: Malvern Zetasizer 1000, Malvern Inst. Limited).

In step A) for the preparation of the polyurethanes used according to the invention, the molar ratio of NCO groups of the compounds from component A1) to NCO-reactive groups such amino, hydroxy or thiol groups of the compounds of components A2) to A4) in the preparation of the NCO-functional prepolymer is preferably 1.05 to 3.5, more preferably 1.2 to 3.0, particularly preferably 1.3 to 2.5.

The amino-functional compounds in stage B) are used in an amount such that the equivalent ratio of isocyanate-reactive amino groups of these compounds to the free isocyanate groups of the prepolymer is preferably 40 to 150%, more preferably between 50 and 125%, particularly preferably between 60 and 120%.

Suitable polyisocyanates of component A1) are in particular the aromatic, araliphatic, aliphatic or cycloaliphatic polyisocyanates of an NCO functionality of in particular 2 known per se to the person skilled in the art.

Examples of such suitable polyisocyanates are 1,4-butylene diisocyanate, 1,6-hexamethylene diisocyanate (HDI), isophorone diisocyanate (IPDI), 2,2,4 and/or 2,4,4-trimethylhexamethylene diisocyanate, the isomeric bis(4,4′-isocyanatocyclohexyl)methanes or mixtures thereof of any desired isomer content, 1,4-cyclohexylene diisocyanate, 1,4-phenylene diisocyanate, 2,4- and/or 2,6-toluoylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI), and alkyl 2,6-diisocyanatohexanoates (lysine diisocyanates) with C1-C8-alkyl groups.

Besides the aforementioned polyisocyanates, modified diisocyanates with uretdione, isocyanurate, urethane, allophanate, biuret, iminooxadiazinedione and/or oxadiazinetrione structure and also nonmodified polyisocyanate with more than 2 NCO groups per molecule, such as, e.g. 4-isocyanatomethyl 1,8-octanediisocyanate (nonane triisocyanate) or triphenylmethane 4,4′,4″-triisocyanate, can also be co-used, proportionately.

These are preferably polyisocyanates or polyisocyanate mixtures of the type specified above with exclusively aliphatically and/or cycloaliphatically bonded isocyanate groups and an average NCO functionality of the mixture of from 2 to 4, more preferably 2 to 2.6 and particularly preferably 2 to 2.4, very particularly preferably 2. A functionality of approximately 2 is preferred since suitable mechanical properties thereby result.

In A1), particular preference is given to using 1,6-hexamethylene diisocyanate, isophorone diisocyanate, the isomeric bis(4,4′-isocyanatocyclohexyl)methanes, and also mixtures thereof.

In A2), polymeric polyols with a number-average molecular weight Mₙ of from preferably 400 to 8000 g/mol, more preferably from 400 to 6000 g/mol and particularly preferably from 600 to 3000 g/mol are used. These preferably have an OH functionality of from 1.5 to 6, particularly preferably from 1.8 to 3, very particularly preferably from 1.9 to 2.1.

The expression “polymeric” polyols means here in particular that the specified polyols have at least two, preferably at least three, repeat units joined together.

Such polymeric polyols are the polyester polyols, polyacrylate polyols, polyurethane polyols, polycarbonate polyols, polyether polyols, polyester polyaciylate polyols, polyurethane polyaciylate polyols, polyurethane polyester polyols, polyurethane polyether polyols, polyurethane polycarbonate polyols and polyester polycarbonate polyols known per se in polyurethane paint technology. These can be used in A2) individually or in any desired mixtures with one another.

The preferably used polyester polyols are the polycondensates, known per se, of di- and optionally tri- and tetraols and di- and optionally tri- and tetracarboxylic acids or hydroxycarboxylic acids or lactones. Instead of the free polycarboxylic acids, it is also possible to use the corresponding polycarboxylic anhydrides or corresponding polycarboxylic acid esters of lower alcohols for the preparation of the polyesters.

Examples of suitable diols are ethylene glycol, butylene glycol, diethylene glycol, triethylene glycol, polyalkylene glycols, such as polyethylene glycol, also 1,2-propanediol, 1,3-propanediol, butanediol(1,3), butanediol(1,4), hexanediol(1,6) and isomers, neopentyl glycol or hydroxypivalic acid neopentyl glycol ester, where hexanediol(1,6) and isomers, butanediol(1,4), neopentyl glycol and hydroxypivalic acid neopentyl glycol ester are preferred. In addition, it is also possible to use polyols such as trimethylolpropane, glycerol, erythritol, pentaerythritol, trimethylolbenzene or trishydroxyethyl isocyanurate.

Dicarboxylic acids which can be used are phthalic acid, isophthalic acid, terephthalic acid, tetra-hydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethyl-succinic acid. The acid source used may also be the corresponding anhydrides.

If the average functionality of the polyols to be esterified is >2, monocarboxylic acids, such as benzoic acid and hexanecarboxylic acid, can also be co-used.

Preferred acids are aliphatic or aromatic acids of the type specified above. Particular preference is given to adipic acid, isophthalic acid and phthalic acid.

Hydroxycarboxylic acids which can be co-used as reaction participants in the preparation of the polyester polyol with terminal hydroxyl groups are, for example, hydroxycaproic acid, hydroxybutyric acid, hydroxydecanoic acid, hydroxystearic acid and the like. Suitable lactones are caprolactone, butyrolactone and homologues. Preference is given to caprolactone.

Particularly preferred according to the invention as component A2) for the preparation of the polyurethanes are polyester polyols with a number-average molecular weight of from 600 to 3000 g/mol, in particular aliphatic polyester polyols based on aliphatic carboxylic acids and aliphatic polyols, in particular based on adipic acid and aliphatic alcohols, such as hexanediol and/or neopentyl glycol.

As component A2), it is likewise possible to use polycarbonates having hydroxyl groups, preferably polycarbonate diols, with number-average molecular weights Mₙ of from preferably 400 to 8000 g/mol, preferably 600 to 3000 g/mol. These are obtainable by reacting carbonic acid derivatives, such as diphenyl carbonate, dimethyl carbonate or phosgene, with polyols, preferably diols.

Examples of such diols are ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentanediol-1,3, dipropylene glycol, polypropylene glycols, dibutylene glycol, polybutylene glycols, bisphenol A and lactone-modified diols of the aforementioned type.

Preferably, the diol component comprises 40 to 100% by weight of hexanediol, preference being given to 1,6-hexanediol and/or hexanediol derivatives. Such hexanediol derivatives are based on hexanediol and, besides terminal OH groups, have ester or ether groups. Such derivatives are obtainable by reacting hexanediol with excess caprolactone or by etherifying hexanediol with itself to give the di- or trihexylene glycol.

Instead of or in addition to pure polycarbonate diols, it is also possible to use polyether-polycarbonate diols in A2).

Polycarbonates having hydroxyl groups are preferably linear in structure.

As component A2), it is likewise possible to use polyether polyols.

Of particular suitability are, for example, the polytetramethylene glycol polyethers known per se in polyurethane chemistry, as are obtainable through polymerization of tetrahydrofuran by means of cationic ring-opening.

Likewise suitable polyether polyols are the addition products, known per se, of styrene oxide, ethylene oxide, propylene oxide, butylene oxide and/or epichlorohydrin onto di- or polyfunctional starter molecules. Thus, in particular polyalkylene glycols, such as polyethylene, polypropylene and/or polybutylene glycols, can be used, in particular those with the aforementioned preferred molecular weights.

Suitable starter molecules which can be used are all of the compounds known according to the prior art, such as, for example, water, butyl diglycol, glycerol, diethylene glycol, trimethyolpropane, propylene glycol, sorbitol, ethylenediamine, triethanolamine, 1,4-butanediol.

Particularly preferred components in A2) are polytetramethylene glycol polyethers and mixtures thereof with other polyols and exclusively polytetramethylene glycol polyethers are particularly preferred.

Particular preference is given to using exclusively polytetramethylene glycol polyethers, but as mixtures of at least two polytetramethylene glycol polyethers with different average molecular weights. Very particular preference is given to mixtures of two fractions of polytetramethylene glycol polyethers, where the fraction with the lower molecular weight is present from 3 to 80% by weight, preferably from 8 to 30% by weight and very particularly preferably from 14 to 25% by weight, based on the total amount of the polytetramethylene glycol polyethers used.

The number-average molecular weight Mₙ of the fraction with the lower molecular weight is preferably in the range from 400 to 2000 g/mol, particularly preferably from 650 to 1400 g/mol, very particularly preferably around 1000 g/mol. The number-average molecular weight Mₙ, of the fraction with the higher molecular weight is preferably in the range from 1000 to 8000 g/mol, particularly preferably from 1500 to 4000 g/mol, very particularly preferably around 2000 g/mol.

In one preferred embodiment of the invention, component A2) accordingly comprises:
at least one poly(tetramethylene glycol) polyether polyol (such as HO—(CH₂—CH₂—CH₂—CH₂—O)ₓ—H),
particularly preferably no other component than poly(tetramethylene glycol) polyether polyols (such as HO—(CH₂—CH₂—CH₂—CH₂—O)ₓ—H),
very particularly preferably two fractions of polytetramethylene glycol polyethers, where the fraction with the lower molecular weight is present to 3 to 80% by weight and preferably to 8 to 30% by weight and very particularly preferably from 14 to 25% by weight, based on the total amount of the polytetramethylene glycol polyethers used and
   the component A), according to the definition, has essentially neither ionic nor ionogenic groups.

The hydroxy-functional compounds optionally used as component A3) expediently have molecular weights of 62 up to less than 400 g/mol. In contrast to components A2), component A3) is in particular not polymeric hydro-functional compounds, but monomeric compounds which have no repeat units like the compounds of component A2).

As component A3), it is possible to use, in particular, polyols, preferably of the specified molecular weight range, preferably having up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A, (2,2-bis(4-hydroxycyclohexyl)propane), trimethylolpropane, glycerol, pentaerythritol and any desired mixtures with one another.

Also suitable as component A3) are esterdiols of the specified molecular weight range, such as α-hydroxybutyl ε-hydroxycaproate, ω-hydroxyhexyl γ-hydroxybutyrate, β-hydroxyethyl adipate or bis(β-hydroxyethyl)terephthalate.

In addition, as component A3) it is also possible to use monofunctional, isocyanate-reactive, hydroxyl-group-containing compounds. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Preferred compounds of component A3) are 1,6-hexanediol, 1,4-butanediol, neopentyl glycol and trimethylolpropane.

In one preferred embodiment of the invention, the polyurethane used according to the invention comprises less than about 10% by weight of component A3), preferably less than 5% by weight of component A3), in each case based on the total mass of the polyurethane, even more preferably component A3) is not used for the preparation of the polyurethane.

Used as component A4) for the preparation of the polyurethanes used according to the invention are optionally one or more isocyanate-reactive hydrophilizing agents, which are preferably selected from anionic, potentially anionic and/or nonionic hydrophilizing agents. The isocyanate-reactive hydrophilizing agents used as component A4) are in particular different from components A2) and A3).

Anionically and/or potentially anionically hydrophilizing compounds of component A4) are understood in particular as meaning all compounds which have at least one isocyanate-reactive group, such as a hydroxyl group or an amino group, and also at least one functionality such as e.g. —COO⁻M⁺, —SO₃⁻M⁺, —PO(O⁻M⁺)₂ where M⁺ is, for example, a metal cation, in particular alkali metal cation, such as Na⁺, H⁺, NH₄⁺, NHR₃⁺, where R can in each case be a C1-C12-alkyl radical, C5-C6-cycloalkyl radical and/or a C2-C4-hydroxyalkyl radical. As is known to the person skilled in the art, upon interaction with aqueous media, these compounds enter into a pH-dependent dissociation equilibrium and can thereby be negatively or neutrally charged. Suitable anionically or potentially anionically hydrophilizing compounds include in particular: mono- and dihydroxy-carboxylic acids, mono- and dihydroxysulphonic acids, and also mono- and dihydroxyphosphonic acids and their salts. Examples of such anionic or potentially anionic hydrophilizing agents are dimethylolpropionic acid, dimethylolbutyric acid, hydroxypivalic acid, malic acid, citric acid, glycolic acid, lactic acid and the propoxylated adduct of 2-butenediol and NaHSO₃, as is described in DE-A 2 446 440, page 5-9, formula I-III. Preferred anionic or potentially anionic hydrophilizing agents of component A4) are those of the type specified above which have carboxylate and/or carboxylic acid groups and/or sulphonate groups.

Particularly preferred anionic or potentially anionic hydrophilizing agents A4) are those which contain carboxylate and/or carboxylic acid groups as ionic or potentially ionic groups, such as dimethylolpropionic acid, dimethylolbutyric acid and hydroxypivalic acid and/or salts thereof.

Suitable nonionically hydrophilizing compounds of component A4) also include in particular polyoxyalkylene ethers, which have isocyanate-reactive groups, such as hydroxy and/or amino groups. They are preferably a monofunctional polyoxyalkylene ether, in particular a monohydroxy-functional polyoxyalkylene ether, such as, for example, a monohydroxy-functional polyether based on ethylene oxide/propylene oxide, such as the polyether LB 25 sold by the applicant.

Examples of the nonionically hydrophilizing compounds as component A4) are the monohydroxy-functional polyalkylene oxide polyether alcohols having, on statistical average, 5 to 70, preferably 7 to 55, ethylene oxide units per molecule, as are accessible in a manner known per se, by alkoxylation of suitable starter molecules, in particular also monofunctional alcohols (e.g. in Ullmann's Encyclopedia of Industrial Chemistry, 4^{th} edition, volume 19, Verlag Chemie, Weinheim pp. 31-38).

These are in particular either pure polyethylene oxide ethers or mixed polyalkylene oxide ethers, in particular polyethylene oxide/polypropylene oxide polyether compounds, which may have a block-like or random structure, where they preferably comprise at least 30 mol %, preferably at least 40 mol %, based on all of the alkylene oxide units present, of ethylene oxide units.

Particularly preferred nonionic compounds are monofunctional mixed polyalkylene oxide polyethers which have 40 to less than 100 mol % of ethylene oxide units and more than 0 to 60 mol % of propylene oxide units.

Suitable starter molecules for such nonionic hydrophilizing agents A4) are in particular saturated monoalcohols, such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as, for example, diethylene glycol monobutyl ether, unsaturated alcohols, such as allyl alcohol, 1,1-dimethylallyl alcohol or oleyl alcohol, aromatic alcohols, such as phenol, the isomeric cresols or methoxyphenols, araliphatic alcohols, such as benzyl alcohol, anisyl alcohol or cinnamyl alcohol. Less preferred are secondary monomamines, such as dimethylamine, diethylamine, dipropylamine, diisopropylamine, dibutylamine, bis(2-ethylhexyl)amine, N-methyl- and N-ethylcyclohexylamine or dicyclohexylamine, and also heterocyclic secondary amines, such as morpholine, pyrrolidine, piperidine or 1H-pyrazole.

Preferred starter molecules are saturated monoalcohols of the aforementioned type. Particular preference is given to using diethylene glycol monobutyl ether or n-butanol as starter molecules.

Alkylene oxides suitable for the alkoxylation reaction are in particular ethylene oxide and propylene oxide, which can be used in the alkoxylation reaction in any desired order or else in a mixture.

If desired, in step B), the component B1) can be used. Component B1) is selected from aminofunctional compounds, preferably having molecular weights of from 32 to 400 g/mol. Component B1) is different from component A4) and components B2). It differs from component A4) and components B2) preferably in that it has no anionic and potentially anionic groups.

Components B1) which can be used are in particular di- or polyamines, such as 1,2-ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, triaminononane, 1,3- and 1,4-xylylenediamine, α,α,α′,α′-tetramethyl-1,3- and -1,4-xylylenediamine and 4,4-diaminodicyclo-hexylmethane and/or dimethylethylenediamine. The use of hydrazine or hydrazides, such as adipic acid dihydrazide, is likewise possible. Preference is given to isophoronediamine, 1,2-ethylene-diamine, 1,4-diaminobutane, hydrazine and diethylenetriamine.

Moreover, compounds which, besides a primary amino group, also have secondary amino groups or besides an amino group (primary or secondary) also have OH groups, can also be used as component B1). Examples thereof are primary/secondary amines, such as diethanolamine, 3-amino-1-methylaminopropane, 3-amino-1-ethylaminopropane, 3-amino-1-cyclohexylamino-propane, 3-amino-1-methylaminobutane, alkanolamines, such as N-aminoethylethanolamine, ethanolamine, 3-aminopropanol, neopentanolamine.

In addition, monofunctional isocyanate-reactive amine compounds can also be used as component B1), such as, for example, methylamine, ethylamine, propylamine, butylamine, octylamine, laurylamine, stearylamine, isononyloxypropylamine, dimethylamine, diethylamine, dipropylamine, dibutylamine, n-methylaminopropylamine, diethyl(methyl)aminopropylamine, morpholine, piperidine, and/or suitable substituted derivatives thereof, amidoamines of diprimary amines and monocarboxylic acids, monoketimes of diprimary amines, primary/tertiary amines, such as N,N-di-methylaminopropylamine.

Preferred compounds of component B1) are 1,2-ethylenediamine, 1,4-diaminobutane and isophoronediamine.

Preferably, in step B), the component B2) can be used. Component B2) is selected from isocyanate-reactive, preferably aminofunctional hydrophilizing agents which are selected preferably from ionic and/or ionogenic, in particular anionic and potentially anionic hydrophilizing agents. These may be the same compounds which can be used, if desired, in step A) as component A4), with the exception of the nonionic hydrophilizing agents. Consequently, identical or different compounds can be used as component A4) and component B2) in a process for the preparation of the polyurethane used according to the invention.

With regard to the anionically or potentially anionically hydrophilizing compounds of component B2), reference can therefore be made per se to the definitions given for component A4). Component B2) includes in particular all compounds which have at least one isocyanate-reactive group, preferably an amino group, and also at least one anionic functionality such as, for example, —COO⁻M⁺, —SO₃⁻M⁺, —PO(O⁻M₊)₂ where M⁺ is, for example, a metal cation, such as alkali metal cation, such as Na⁺, H⁺, NH₄⁺, NHR₃⁺, where R can in each case be a C1-C12-alkyl radical, C5-C6-cycloalkyl radical and/or a C2-C4-hydroxyalkyl radical. As is known to the person skilled in the art, upon interaction with aqueous media, these groups enter into a pH-dependent dissociation equilibrium and can thereby be negatively or neutrally charged.

Anionically or potentially anionically hydrophilizing compounds suitable particularly as component B2) are mono- and diaminocarboxylic acids, mono- and diaminosulphonic acids and also mono- and diaminophosphonic acids and their salts. Examples of such anionic and potentially anionic hydrophilizing agents are N-(2-aminoethyl)β-alanine, 2-(2-aminoethylamino)ethane-sulphonic acid, ethylenediaminepropyl- or -butylsulphonic acid, 1,2- or 1,3-propylenediamine-β-ethylsulphonic acid, glycine, alanine, taurine, lysine, 3,5-diaminobenzoic acid and the addition product of IPDA and acrylic acid (EP-A 0 916 647, Example 1). Furthermore, the cyclohexylaminopropanesulphonic acid (CAPS) known from WO-A 01/88006 can be used as anionic or potentially anionic hydrophilizing agent.

Preferred anionic or potentially anionic hydrophilizing agents of component B2) are those of the type specified above which have carboxylate and/or carboxylic acid groups and/or sulphonate groups, such as the salts of N-(2-aminoethyl)-β-alanine, of 2-(2-aminoethylamino)ethansulphonic acid or of the addition product of IPDA and acrylic acid (EP-A 0 916 647, Example 1).

For the hydrophilization, it is also possible to use mixtures of anionic and/or potentially anionic hydrophilizing agents and nonionic hydrophilizing agents.

The polyurethane used according to the invention preferably comprises an anionic and/or potentially anionic hydrophilizing agent as component A4) (then it is already reacted during the prepolymer formation) or as component B2) (then it is not used until during the reaction of the prepolymer). Preferably, during the reaction of the prepolymer in step B), an anionic and/or potentially anionic hydrophilizing agent is added as component B2). In other words, in one preferred embodiment, the polyurethane used according to the invention comprises an anionic group or a potentially anionic group, i.e. one dissociatable into a polyurethane anion. The particularly preferred anionic or potentially anionic group is the sulphonate group: —SO₃⁻M⁺. This is particularly preferably introduced into the polyurethane by means of a diaminosulphonate, in particular by means of NH₂—CH₂CH₂—NH—CH₂CH₂—SO₃Na.

In one preferred embodiment, both component B1) and also component B2) are used for the preparation of the special polyurethane dispersions. Through the use of component B1) it is possible to build up a high molecular mass without the viscosity of the isocyanate-functional prepolymer prepared previously increasing to an extent which would hinder processing. Through the use of the combination of components B1) and B2) it is possible in particular to establish an optimum balance between hydrophilicity and chain length.

In a preferred embodiment for the preparation of the special polyurethane dispersions, the components A1) to A4) and B1) to B2) are used in the following amounts, the individual amounts always adding up to 100% by weight:

5 to 40% by weight of component A1),
40 to 90% by weight of A2),
0.5 to 20% by weight of the sum of components A3) and/or B1)
0.1 to 25% by weight of the sum of components A4) and/or B2), where, based on the total amounts of components A1) to A4) and B1) to B2), particularly preferably 0.1 to 5% by weight of anionic and/or potentially anionic hydrophilizing agents from A4) and/or B2) are used.

In a particularly preferred embodiment for the preparation of the special polyurethane dispersions, the components A1) to A4) and B1) to B2) are used in the following amounts, with the individual amounts always adding up 100% by weight:

5 to 35% by weight of component A1),
60 to 90% by weight of A2),
0.5 to 15% by weight of the sum of components A3) and/or B1)
0.1 to 15% by weight of the sum of the components A4) and/or B2), where, based on the total amounts of the components A1) to A4) and B1) to B2), 0.2 to 4% by weight of anionic or potentially anionic hydrophilizing agents from A4) and/or B2) are used.

In a very particularly preferred embodiment for the preparation of the special polyurethane dispersions, the components A1) to A4) and B1) to B2) are used in the following amounts, with the individual amounts always adding to 100% by weight:

10 to 30% by weight of component A1),
65 to 85% by weight of A2),
0.5 to 14% by weight of the sum of components A3) and/or B1),
0.1 to 13.5% by weight of the sum of the components A4) and/or B2), where, based on the total amounts of components A1) to A4) and B1) to B2), 0.5 to 3.0% by weight of anionic and/or potentially anionic hydrophilizing agents from A4) and/or B2) are used.

The preparation of the preferably anionically hydrophilized polyurethane dispersions can preferably be carried out in one or more stage(s) in homogeneous phase, or in the case of a multistage reaction, sometimes in disperse phase. Following completely or partially carried-out polyaddition in step A) (components A1) to A4)), a dispersion, emulsification or dissolution step preferably takes place. Afterwards, a further polyaddition or modification in disperse phase optionally takes place.

Here, all of the processes known from the prior art, such as, for example, prepolymer mixing process, acetone process or melt dispersion process, can be used. Preference is given to using the acetone process.

For the preparation according to the acetone process, usually the constituents A2) to A4) and the polyisocyanate component A1) are completely or partially initially introduced for the preparation of an isocyanate-functional polyurethane prepolymer and optionally diluted with a solvent which is miscible with water but inert towards isocyanate groups, and heated to temperatures in the range from 50 to 120° C. To increase the rate of the isocyanate addition reaction, the catalysts known in polyurethane chemistry can be used.

Suitable solvents are the customary aliphatic, ketofunctional solvents such as acetone, 2-butanone, which can be added not only at the start of the preparation, but optionally in parts also later on. Preference is given to acetone and 2-butanone.

Other solvents such as xylene, toluene, cyclohexane, butyl acetate, methoxypropyl acetate, N-methylpyrrolidone, N-ethylpyrrolidone, solvents with ether or ester units can additionally be used and completely or partially distilled off or remain in their entirety in the dispersion in the case of N-methylpyrrolidone, N-ethylpyrrolidone. However, apart from the customary aliphatic, ketofunctional solvents, preferably no other solvents are used. Then, any constituents of A1) to A4) not added at the start of the reaction are metered in.

In the preparation of the polyurethane prepolymer from A1) to A4), the ratio of quantitative amounts of isocyanate groups to isocyanate-reactive groups is preferably 1.05 to 3.5, more preferably 1.2 to 3.0, particularly preferably 1.3 to 2.5. Preferably, thus, isocyanate-functional prepolymers are formed which are then preferably reacted with aminofunctional compounds B1) and/or B2).

The reaction of components A1) to A4) to give the prepolymer takes place partially or completely, but preferably completely. Thus, preferably polyurethane prepolymers which contain free isocyanate groups are obtained without a diluent or in solution.

In the neutralization step for the partial or complete conversion of potentially anionic groups to anionic groups, use is made of bases such as tertiary amines, e.g. trialkylamines having 1 to 12, preferably 1 to 6, carbon atoms, particularly preferably 2 to 3, carbon atoms in each alkyl radical or alkali metal bases such as the corresponding hydroxides.

Examples thereof are trimethylamine, triethylamine, methyldiethylamine, tripropylamine, N-methylmorpholine, methyldiisopropylamine, ethyldiisopropylamine and diisopropylethylamine. The alkyl radicals can, for example, also carry hydroxyl groups, as in the case of the dialkylmonoalkanol-, alkyldialkanol- and trialkanolamines. Neutralizing agents which can be used are optionally also inorganic bases, such as aqueous ammonia solution or sodium or potassium hydroxide.

Preference is given to ammonia, triethylamine, triethanolamine, dimethylethanolamine or diisopropylethylamine and also sodium hydroxide and potassium hydroxide, and particular preference is given to sodium hydroxide and potassium hydroxide.

The quantitative amount of the bases is between 50 and 125 mol %, preferably between 70 and 100 mol % of the quantitative amount of the acid groups to be neutralized. The neutralization can also take place at the same time as the dispersion by the dispersion water already comprising the neutralizing agent.

Afterwards, in a further process step, if it has not yet occurred or has only partly occurred, the resulting prepolymer is dissolved preferably with the help of aliphatic ketones such as acetone or 2-butanone.

During the chain extension/termination in stage B), NH₂— and/or NH-functional components are partially or completely reacted with the still present isocyanate groups of the prepolymer. Preferably, the chain extension/termination is carried out prior to the dispersion in water.

For the chain termination, amines B1) with a group which is reactive towards isocyanates, such as methylamine, ethylamine, propylamine, butylamine, octylamine, laurylamine, stearylamine, isononyloxypropylamine, dimethylamine, diethylamine, dipropylamine, dibutylamine, N-methyl-aminopropylamine, diethyl(methyl)aminopropylamine, morpholine, piperidine, and suitable substituted derivatives thereof, amidoamines of diprimary amines and monocarboxylic acids, monoketimes of diprimary amines, primary/tertiary amines, such as N,N-dimethyl-aminopropylamine, are usually used.

If anionic or potentially anionic hydrophilizing agents corresponding to the definition B2) with NH₂ or NH groups are used for the partial or complete chain extension, the chain extension of the prepolymers preferably takes place before the dispersion.

The aminic components B1) and B2) can optionally be used in water- or solvent-diluted form in the process according to the invention individually or in mixtures, where in principle any order of the addition is possible.

If water or organic solvents are co-used as diluents, then the diluent content in the component used in B) for the chain extension is preferably 70 to 95% by weight.

The dispersion preferably takes place after the chain extension. For this, the dissolved and chain-extended polyurethane polymer is introduced optionally with high shear, such as, for example vigorous stirring, either into the dispersion water or, vice versa, the dispersion water is stirred into the chain-extended polyurethane polymer solutions. Preferably, the water is added to the dissolved chain-extended polyurethane polymer.

The volatile solvent still present in the dispersions after the dispersion step is then usually removed by distillation. Removal even during dispersion is likewise possible.

Following complete reaction of the isocyanate groups or following dispersion, alcohol-functional solvents can also be added, for example as coalescing agents. Examples are ethanol, n-butanol, n-propanol, ethylene glycol monobutyl ether (2-butoxyethanol), diethylene glycol monomethyl ether, propylene glycol monomethyl ether, propylene glycol monobutyl ether, dipropylene glycol monobutyl ether and tripropylene glycol monobutyl ether.

The polyurethane dispersions preferably used according to the invention are generally aqueous dispersions. The pH of the polyurethane dispersions used according to the invention is typically less than 9.0, preferably less than 8.5, particularly preferably less than 8.0 and is very particularly preferably 6.0 to 7.5.

The solids content of the polyurethane dispersions which is preferably used for the preparation of the cosmetic composition of the invention is generally 20 to 70, preferably 30 to 65, particularly preferably 35 to 60% by weight. The solids contents are determined by heating a weighed sample to 125° C. to constant weight. At constant weight, the solids content is calculated by reweighing the sample.

Advantageously, the cosmetic compositions according to the invention for colouring hair may be present as shampoo, gels, cream, foam aerosol, emulsions or aqueous or aqueous-alcoholic solutions.

The cosmetic compositions according to the invention comprise preferably 0.1 to 20% by weight of the polyurethane dispersions essential to the invention, particularly preferably 0.5 to 10% by weight, based on the total composition.

Besides the polyurethane dispersions essential to the invention, the cosmetic compositions according to the invention comprise one or more direct dyes and/or at least one oxidation dye precursor, preferably a mixture of at least one developer substance and at least one coupler substance.

“Direct dyes” are understood as meaning already developed pigments and dyes. Their colour development is already present and does not have to be brought about beforehand, e.g. by oxidation processes.

The direct dyes which can be used according to the invention are selected from the neutral, acidic or cationic nitrated benzene dyes, neutral, acidic or cationic direct azo dyes, neutral, acidic or cationic direct quinone dyes and in particular anthraquinone dyes, direct azine dyes, direct triarylmethane dyes, direct indoamine dyes and direct natural dyes.

Preferred direct benzene dyes are those selected from the group consisting of:
1,4-diamino-2-nitrobenzene,
1-amino-2-nitro-4-β-hydroxyethylaminobenzene,
1-amino-2-nitro-4-bis(β-hydroxyethyl)aminobenzene,
1,4-bis(β-hydroxyethylamino)-2-nitrobenzene,
1-β-hydroxyethylamino-2-nitro-4-bis(β-hydroxyethylamino)benzene,
1-β-hydroxyethylamino-2-nitro-4-aminobenzene,
1-β-hydroxyethylamino-2-nitro-4(ethyl)(β-hydroxyethyl)aminobenzene,
1-amino-3-methyl-4-β-hydroxyethylamino-6-nitrobenzene,
1-amino-2-nitro-4-β-hydroxyethylamino-5-chlorobenzene,
1,2-diamino-4-nitrobenzene,
1-amino-2-β-hydroxyethylamino-5-nitrobenzene,
1,2-bis(β-hydroxyethylamino)-4-nitrobenzene,
1-amino-2-tris(hydroxymethyl)methylamino-5-nitrobenzene,
1-hydroxy-2-amino-5-nitrobenzene,
1-hydroxy-2-amino-4-nitrobenzene,
1-hydroxy-3-nitro-4-aminobenzene,
1-hydroxy-2-amino-4,6-dinitrobenzene,
1-β-hydroxyethyloxy-2-β-hydroxyethylamino-5-nitrobenzene,
1-methoxy-2-β-hydroxyethylamino-5-nitrobenzene,
1-β-hydroxyethyloxy-3-methylamino-4-nitrobenzene,
1-β,γ-dihydroxypropyloxy-3-methylamino-4-nitrobenzene,
1-β-hydroxyethylamino-4-β,γ-dihydroxypropyloxy-2-nitrobenzene,
1-β,γ-dihydroxypropylamino-4-trifluoromethyl-2-nitrobenzene,
1-β-hydroxyethylamino-4-trifluoromethyl-2-nitrobenzene,
1-β-hydroxyethylamino-3-methyl-2-nitrobenzene,
1-β-aminoethylamino-5-methoxy-2-nitrobenzene,
1-hydroxy-2-chloro-6-ethylamino-4-nitrobenzene,
1-hydroxy-2-chloro-6-amino-4-nitrobenzene,
1-hydroxy-6-bis(β-hydroxyethyl)amino-3-nitrobenzene,
1-β-hydroxyethylamino-2-nitrobenzene and
1-hydroxy-4-β-hydroxyethylamino-3-nitrobenzene.

For example, the direct azo dyes can be the cationic azo dyes which are described in the patent applications WO 95/15144, WO 95/01772 and EP 714 954.

Further advantageous are the following direct azo dyes: Disperse Red 17, Acid Yellow 9, Acid Black 1, Basic Red 22, Basic Red 76, Basic Yellow 57, Basic Brown 16, Acid Yellow 36, Acid Orange 7, Acid Red 33, Acid Red 35, Basic Brown 17, Acid Yellow 23, Acid Orange 24, Disperse Black 9,1-(4′-diaminodiphenylazo)-2-methyl-4-bis(β-hydroxyethyl)aminobenzene and 4-hydroxy-3-(2-methoxyphenylazo)-1-naphthalenesulphonic acid, where the aforementioned trivial names refer to compounds according to the International Cosmetic Ingredient Dictionary and Handbook, 12th Edition, 2008 by Tara E. Gottschalck and John E. Bailey, Ph.D. (Editor).

Preferred direct quinone dyes are those selected from the group consisting of:
Disperse Red 15,
Solvent Violet 13,
Acid Violet 43,
Disperse Violet 1,
Disperse Violet 4,
Disperse Blue 1,
Disperse Violet 8,
Disperse Blue 3,
Disperse Red 11,
Acid Blue 62,
Disperse Blue 7,
Basic Blue 22,
Disperse Violet 15,
Basic Blue 99,
   where the aforementioned trivial names refer to compounds according to the International Cosmetic Ingredient Dictionary and Handbook, 12th Edition, 2008 by Tara E. Gottschalck and John E. Bailey, Ph.D. (Editor),
   and also the following compounds:
1-N-methylmorpholiniumpropylamino-4-hydroxyanthraquinone,
1-aminopropylamino-4-methylaminoanthraquinone,
1-aminopropylaminoanthmquinone,
5-β-hydroxyethyl-1,4-diaminoanthraquinone,
2-aminoethylaminoanthraquinone and
1,4-bis(β,γ-dihydroxypropylamino)anthraquinone.

Of the azine dyes, the following compounds are to be mentioned:
Basic Blue 17,
Basic Red 2,
   according to the International Cosmetic Ingredient Dictionary and Handbook, 12th Edition, 2008 by Tara E. Gottschalck and John E. Bailey, Ph.D. (Editor).

Preferred triarylmethane dyes are those selected from the group consisting of:
Basic Green 1,
Acid Blue 9,
Basic Violet 3,
Basic Violet 14,
Basic Blue 7,
Acid Violet 49,
Basic Blue 26,
Acid Blue 7,
   according to the International Cosmetic Ingredient Dictionary and Handbook, 12th Edition, 2008 by Tara E. Gottschalck and John E. Bailey, Ph.D. (Editor).

Preferred indoamine dyes are those selected from the group consisting of:
2-β-hydroxyethylamino-5-[bis(β-4′-hydroxyethyl)amino]-anilino-1,4-benzoquinone,
2-β-hydroxyethylamino-5-(2′-methoxy-4′-amino)anilino-1,4-benzoquinone,
3-N-(2′-chloro-4′-hydroxy)phenylacetylamino-6-methoxy-1,4-benzoquinonimine,
3-N-(3′-chloro-4′-methylamino)phenylureido-6-methyl-1,4-benzoquinonimine,
3-[4′-N-(ethylcarbamoylmethyl)amino]phenylureido-6-methyl-1,4-benzoquinonimine.

Natural direct dyes are selected from the group lawsone, juglone, alizarin, purpurin, carminic acid, kermesic acid, purpurogallin, protocatechaldehyde, indigo, isatin, curcumin, spinulosin and apigenidin. Extracts or decoctions can also be used which contain natural dyes, such as, for example, cataplasms or extracts based on henna.

If direct dyes are used for the colouring, then their fraction in the cosmetic compositions according to the invention is typically up to 20% by weight and preferably up to 10% by weight, based on the total weight of the compositions.

If oxidation dye precursors are used for the colouring, then these are preferably composed of a developer substance and a coupler substance.

Suitable developer substances are 4-diaminobenzene, 2,5-diaminotoluene, tetraminopyrimidines, triaminohydroxypyrimidines, 1,2,4-triaminobenzene, 2-(2,5-diaminophenyl)ethanol, 2-(2′-hydroxy-ethylamino)-5-aminotoluene and 1-amino-4-bis-(2′-hydroxyethyl)aminobenzene and water-soluble salts thereof; exemplary coupler substances are resorcinol, 2-methylresorcinol, 4-chlororesorcinol, 2-amino-4-chlorophenol, 4-(N-methyl)aminophenol, 2-aminophenol, 3-aminophenol, 1-methyl-2-hydroxy-4-aminobenzene, 3-N,N-dimethylaminophenol, 4-amino-3-methylphenol, 5-amino-2-methylphenol, 6-amino-3-methylphenol, 3-amino-2-methylamino-6-methoxypyridine, 2-amino-3-hydroxypyridine, 4-aminodiphenylamine, 4,4′-diaminodiphenylamine, 2-dimethylamino-5-aminopyridine, 2,6-diaminopyridine, 1,3-diaminobenzene, 1-amino-3-(2′-hydroxyethylamino)benzene, 1-amino-3-[bis(2′-hydroxyethyl)amino]benzene, 1,3-diaminotoluene, α-naphthol, 1,4-diamino-2-chlorobenzene, 4,6-dichlororesorcinol, 4-hydroxy-1,2-methylenedioxybenzene, 1,5-dihydroxynaphthalene, 1,7-dihydroxynaphthalene, 2,7-dihydroxy-naphthalene, 1-hydroxynaphthalene, 4-hydroxy-1,2-methylenedioxybenzene, 2,4-diamino-3-chlorophenol, and/or 1-methoxy-2-amino-4-(2′-hydroxyethylamino)benzene.

Exemplary coupler substances are resorcinol, 2-methylresorcinol, 4-chlororesorcinol, 2-amino-4-chlorophenol, 4-(N-methyl)aminophenol, 2-aminophenol, 3-aminophenol, 1-methyl-2-hydroxy-4-aminobenzene, 3-N,N-dimethylaminophenol, 5-amino-2-methylphenol, 6-amino-3-methylphenol, 3-amino-2-methylamino-6-methoxypyridine, 2-amino-3-hydroxypyridine, 2-dimethylamino-5-aminopyridine, 2,6-diaminopyridine, 1,3-diaminobenzene, 1-amino-3-(2′-hydroxyethyl-amino)benzene, 1-amino-3-[bis(2′-hydroxyethyl)amino]benzene, α-naphthol, 4,6-dichloro-resorcinol, 1,3-diaminotoluene, 1-hydroxynaphthalene, 4-hydroxy-1,2-methylenedioxybenzene, 1,5-dihydroxynaphthalene, 1,7-dihydroxynaphthalene, 2,7-dihydroxynaphthalene, 4-hydroxy-1,2-methylenedioxybenzene, 2,4-diamino-3-chlorophenol and/or 1-methoxy-2-amino-4-(2′-hydroxyethylamino)benzene and water-soluble salts thereof.

If oxidation dye precursors are used for the colouring, developer substances and coupler substances are preferably present in the molar ratio 1:3 to 5:1, particularly preferably 1:1 to 3:1.

If oxidation dye precursors are used for the colouring, then the use concentration of developer substances and coupler substances is typically up to 5% by weight, based on the total weight of the composition, depending on the desired coloration.

The cosmetic compositions according to the invention can also preferably comprise thickeners.

Advantageous thickeners are:
crosslinked or uncrosslinked acrylic acid or methacrylic acid homopolymers or copolymers. These include crosslinked homopolymers of methacrylic acid or acrylic acid, copolymers of acrylic acid and/or methacrylic acid and monomers which are derived from other acrylic or vinylic monomers, such as C10-30 alkyl acrylates, C10-30-alkyl methacrylates and vinyl acetate.
Thickening polymers of natural origin, for example based on cellulose, guar gum, xanthan, scleroglucan, gellan gum, rhamsan and karaya gum, alginates, maltodextrin, starch and its derivatives, carob seed flour, hyaluronic acid.
Nonionic, anionic, cationic or amphoteric associative polymers, e.g. based on polyethylene glycols and their derivatives, or polyurethanes.
Crosslinked or uncrosslinked homopolymers or copolymers based on acrylamide or methacrylamide, such as homopolymers of 2-acrylamido-2-methylpropanesulphonic acid, copolymers of acrylamide or methacrylamide and methacryloyloxyethyltrimethyl-ammonium chloride or copolymers of acrylamide and 2-acrylamido-2-methyl-propanesulphonic acid.

Particularly preferred thickeners are those of natural origin, crosslinked acrylic acid or methacrylic acid homopolymers or copolymers and crosslinked copolymers of 2-acrylamido-2-methylpropanesulphonic acid of the aforementioned type.

Very particularly preferred thickeners are xanthan gum, such as the products supplied under the names Keltrol® and Kelza® by CP Delco or the products from RHODIA with the name Rhodopol and guar gum, such as the products available under the name Jaguar® HP105 from RHODIA.

Likewise very particularly preferred thickeners are crosslinked homopolymers of methacrylic acid or acrylic acid, which are commercially available from Lubrizol under the names Carbopol® 940, Carbopol® 941, Carbopol® 980, Carbopol® 981, Carbopol® ETD 2001, Carbopol® EDT 2050, Carbopol® 2984, Carbopol® 5984 and Carbopol® Ultrez 10, from 3V under the names Synthalen® K, Synthalen® L and Synthalen® MS and from PROTEX under the names Modarez® V 1250 PX, Modarez® V2000 PX, Viscaron® A1600 PE and Viscaron® A700 PE.

Likewise very particularly preferred thickeners are crosslinked copolymers of acrylic acid or methacrylic acid and a C₁₀₋₃₀-alkyl acrylate or C₁₀₋₃₀-alkyl methacrylate and copolymers of acrylic acid or methacrylic acid and vinylpyrrolidone. Such copolymers are commercially available, for example, from Lubrizol under the names Carbopol® 1342, Carbopol® 1382, Pemulen® TR1 or Pemulen® TR2 and from ISP under the names Ultrathix P-100 (INCI: Acrylic Acid/VP Crosspolymer).

Likewise very particularly preferred thickeners are crosslinked copolymers of 2-acrylamido-2-methylpropanesulphonic acid. Such copolymers are available, for example, from Clariant under the names Aristo ex AVC (INCI: Ammonium Acryloyldimethyltaurate/VP Copolymer).

If the thickeners are used, they are typically present in a concentration of up to 2% by weight, preferably up to 1% by weight, based on the total weight of the composition.

It is in some cases advantageous to neutralize the anionic polymeric thickeners, if used as thickeners, to improve their solubility in water and/or their dispersibility in water using suitable bases.

The following bases can be used as neutralizing agents for polymers which contain acid groups: hydroxides whose cation is an ammonium or an alkali metal, such as, for example, NaOH or KOH.

Other neutralizing agents are primary, secondary or tertiary amines, amino alcohols or ammonia. Preference is given here to 2-amino-2-methyl-1,3-propanediol (AMPD), 2-amino-2-ethyl-1,3-propanediol (AEPD), 2-amino-2-methyl-1-propanol (AMP), 2-amino-1-butanol (AB), 2-amino-1,3-propanediol, monoethanolamine (MEA), diethanolamine (DEA), triethanolamine (TEA), monoisopropanolamine (MIPA), diisopropanolamine (DIPA), triisopropanolamine (TIPA), dimethyl laurylamine (DML), dimethyl myristalamine (DMM), and dimethyl stearamine (DMS).

In addition, the cosmetic compositions according to the invention comprise water and optionally a cosmetically suitable solvent. Preferred cosmetically suitable solvents are aliphatic alcohols having C2-4 carbon atoms, such as ethanol, isopropanol, t-butanol, n-butanol; polyol such as propylene glycol, glycerol, ethylene glycol and polyol ethers; acetone; unbranched or branched hydrocarbons, such as pentane, hexane, isopentane and cyclic hydrocarbons such as cyclopentane and cyclohexane; and mixtures thereof.

Furthermore, haircare active ingredients can be used in the cosmetic compositions according to the invention. Care substances which can be used are preferably cyclic polydimethylsiloxanes (cyclomethicones) in concentrations of from 0 to 1.0% by weight of the total formulation or silicone surfactants (polyether-modified siloxanes) of the dimethicone copolyol or simethicone type in concentrations of 0 to 1.0% by weight of the total weight of the composition.

Cyclomethicones are supplied, inter alia, under the trade name Abil®K4 by Goldschmidt or e.g. DC 244, DC 245 or DC 345 by Dow Corning. Dimethicone copolyols are supplied, for example under the trade name DC 193 by Dow Corning and Belsil® DM 6031 by Wacker.

Furthermore, conventional additives may be present in the cosmetic compositions according to the invention in order, for example, to impart certain modifying properties to the composition.

These may be silicones or silicone derivatives, wetting agents, humectants, softeners such as glycerol, glycol and phthalic esters and ethers, fragrances and perfumes, UV absorbers, dyes, pigments and other colorants, anticorrosive agents, neutralizing agents, antioxidants, detackifiers, combining agents and conditioners, antistatic agents, lustre agents, preservatives, proteins and derivatives thereof, amino acids, vitamins, emulsifiers, surface-active agents, viscosity modifiers, thickeners and rheology modifiers, gelling agents, opacifiers, stabilizers, surfactants, sequestrants, complexing agents, pearlizing agents, aesthetic enhancers, fatty acids, fatty alcohols, triglycerides, botanic extracts, clarifying auxiliaries and film formers.

If present, such additives are typically present in amounts of from 0.001% by weight to 15% by weight, preferably 0.01% by weight to 10% by weight, based on the total weight of the cosmetic composition.

The pH of the cosmetic compositions according to the invention can be both in the alkaline range, i.e. from 7.1 to 11, in the neutral range or weakly acidic range, i.e. from 5 to 6.9.

The cosmetic compositions according to the invention can advantageously be present in the form of a lotion, a cream or a foam and be applied by manual spreading. A spray application by means of pump-spray or aerosol, however, is also possible.

The cosmetic compositions according to the invention can be advantageously foamed using a propellant gas.

Accordingly, the cosmetic compositions according to the invention in the form of a lotion, a cream or a foam are likewise provided by the invention. Moreover, the invention provides pump-spray, aerosol packaging and foam dispensers based on pump-spray or aerosol packaging which contain the hair colorant composition according to the invention.

### EXAMPLES

Unless denoted otherwise, all of the percentage data are based on the weight.

Unless noted otherwise, all of the analytical measurements are based on measurements at temperatures of 23° C.

The solids or solid-body contents are determined by heating a weighed sample to 125° C. to constant weight. At constant weight, the solids content is calculated by reweighing.

Unless expressly mentioned othersie, NCO contents were determined volumetrically in accordance with DIN-EN ISO 11909.

The control on free NCO groups was carried out by means of IR spectroscopy (band at 2260 cm⁻¹).

The stated viscosities were determined by means of rotary viscometry in accordance with DIN 53019 at 23° C. using a rotary viscometer from Anton Paar Germany GmbH, Ostfildern, Germany.

The determination of the average particle sizes (the number-average is stated) of the polyurethane dispersions was carried out following dilution with deionized water by means of laser correlation spectroscopy (instrument: Malvern Zetasizer 1000, Malvern Inst. Limited).

### Substances Used and Abbreviations:

Diaminosulphonate: NH₂—CH₂CH₂—NH—CH₂CH₂—SO₃Na (45% strength in water)
Desmophen® 2020/C2200: polycarbonate polyol, OH number 56 mg KOH/g, number-average molecular weight 2000 g/mol (Bayer MaterialScience AG, Leverkusen, Germany)
PolyTHF® 2000: polytetramethylene glycol polyol, OH number 56 mg KOH/g, number-average molecular weight 2000 g/mol (BASF AG, Ludwigshafen, Germany)
PolyTHF® 1000: polytetramethylene glycol polyol, OH number 112 mg KOH/g, number-average molecular weight 1000 g/mol (BASF AG, Ludwigshafen, Germany)
Polyether LB 25: monofunctional polyether based on ethylene oxide/propylene oxide of number-average molecular weight 2250 g/mol, OH number 25 mg KOH/g (Bayer MaterialScience AG, Leverkusen, Germany)

### Example 1

### Polyurethane Dispersion 1

987.0 g of PolyTHF® 2000 (component A2)), 375.4 g of PolyTHF® 1000 (component A2)), 761.3 g of Desmophen® C2200 (component A2)) and 44.3 g of Polyether LB 25 (component A4)) were heated to 70° C. in a standard stirred apparatus. A mixture of 237.0 g of hexamethylene diisocyanate (component A1)) and 313.2 g of isophorone diisocyanate (component A1)) was then added and the mixture was stirred at 120° C. until the theoretical NCO value was reached. The finished prepolymer was dissolved with 4830 g of acetone and in so doing cooled to 50° C., and then a solution of 25.1 g of ethylenediamine (component B1)), 116.5 g of isophoronediamine (component B1)), 61.7 g of diaminosulphonate (component B2)) and 1030 g of water was metered in. The afterstirring time was 10 min. The mixture was then dispersed by adding 1250 g of water. Removal of the solvent followed through distillation in vacuo.

The resulting white dispersion had the following properties:

| Solids content: | 61% | |
|---|---|---|
| Particle size (LCS): | 312 | nm |
| Viscosity (viscometer, 23° C.): | 241 | mPas |

| | |
|---|---|
| pH (23° C.): | 6.02 |
| pH (23° C.): | 7.15 |

### Example 2

### Polyurethane Dispersion 2

450 g of PolyTHF® 1000 (component A2)) and 2100 g of PolyTHF® 2000 (component A2)) were heated to 70° C. Then, a mixture of 225.8 g of hexamethylene diisocyanate (component A1)) and 298.4 g of isophorone diisocyanate (component A1)) was added and the mixture was stirred at 100-115° C. until the actual NCO value had fallen below the theoretical NCO value. The finished prepolymer was dissolved with 5460 g of acetone at 50° C., and then a solution of 29.5 g of ethylenediamine (component B1)), 143.2 g of diaminosulphonate (component B2)) and 610 g of water was metered in. The afterstirring time was 15 min. The mixture was then dispersed by adding 1880 g of water. Removal of the solvent followed through distillation in vacuo and a storage-stable dispersion was obtained.

| Solids content: | 56% | |
|---|---|---|
| Particle size (LCS): | 276 | nm |
| Viscosity: | 1000 | mPas |

### Example 3

### Polyurethane Dispersion 3

1649.0 g of a polyester of adipic acid, hexanediol and neopentyl glycol with an average molecular weight of 1700 g/mol (component A2)) were heated to 65° C. Then, 291.7 g of hexamethylene diisocyanate (component A1)) were added and the mixture was stirred at 100-115° C. until the actual NCO value had fallen below the theoretical NCO value. The finished prepolymer was dissolved with 3450 g of acetone at 50° C., and then a solution of 16.8 g of ethylenediamine (component B1)), 109.7 g of diaminosulphonate (component B2)) and 425 g of water was metered in. The afterstirring time was 15 min. The mixture was then dispersed by adding 1880 g of water. Removal of the solvent followed through distillation in vacuo and a storage-stable dispersion was obtained.

| Solids content: | 42% | |
|---|---|---|
| Particle size (LCS): | 168 | nm |
| Viscosity: | 425 | mPas |

| | |
|---|---|
| pH: | 7.07 |

### Example 4

### Polyurethane Dispersion 4

340 g of a polyester of adipic acid, hexanediol and neopentyl glycol with an average molecular weight of 1700 g/mol (component A2)) were heated to 65° C. Then, 60.1 g of hexamethylene diisocyanate (component A1)) were added and the mixture was stirred at 105° C. until the actual NCO value had fallen below the theoretical NCO value. The finished prepolymer was dissolved with 711 g of acetone at 50° C., and then a solution of 2.1 g of ethylenediamine (component B1)), 32.4 g of diaminosulphonate (component B2)) and 104.3 g of water was metered in. The afterstirring time was 15 min. The mixture was then dispersed by adding 1880 g of water. Removal of the solvent followed through distillation in vacuo and a storage-stable dispersion was obtained.

| Solids content: | 40% | |
|---|---|---|
| Particle size (LCS): | 198 | nm |
| Viscosity: | 700 | mPas |

| | |
|---|---|
| pH: | 6.31 |

### Example 5

### Polyurethane Dispersion 5

450 g of PolyTHF® 1000 (component A2)) and 2100 g of PolyTHF® 2000 (component A2)) were heated to 70° C. Then, a mixture of 225.8 g of hexamethylene diisocyanate (component A1)) and 298.4 g of isophorone diisocyanate (component A1)) was added and the mixture was stirred at 100-115° C. until the actual NCO value had fallen below the theoretical NCO value. The finished prepolymer was dissolved with 5460 g of acetone at 50° C. and then a solution of 351 g of diaminosulphonate (component B2)) and 610 g of water was metered in. The afterstirring time was 15 min. The mixture was then dispersed by adding 1880 g of water. Removal of the solvent followed through distillation in vacuo and a storage-stable dispersion was obtained.

| | |
|---|---|
| Solids content: | 42% |
| Viscosity: | 1370 mPas |

### Application Examples

### Temporary Direct Colorant

| | 1 | 2 | 3 |
|---|---|---|---|
| | % by wt. | % by wt. | % by wt. |
| Polyurethane according to the | 10 | 2.0 | 1.0 |
| invention | | | |
| Ethanol | | 30.0 | |
| Basic Blue 99 (CI 56059) | 0.10 | 0.035 | 0.10 |
| Basic Brown 16 (CI 12250) | 0.20 | 0.005 | 0.20 |
| Basic Brown 17 (CI 12251) | 0.40 | 0.5 | 0.30 |
| Basic Yellow 57 (CI 12719) | 0.15 | | 0.35 |
| Xanthan gum | | | 2.00 |
| Preservative | q.s. | q.s. | q.s. |
| Perfume | q.s. | q.s. | q.s. |
| Water | ad 100 | ad 100 | ad 100 |

### Permanent Colorant

| | % by wt. | % by wt. |
|---|---|---|
| Polyurethane according to the invention | 2.0 | 5.0 |
| Cetyl alcohol | 5.0 | 5.0 |
| Stearyl alcohol | 2.0 | 2.0 |
| Lanolin | 1.5 | 1.5 |
| PEG-20 stearate | 1.5 | 1.5 |
| Oleth-5 | 1.0 | 1.0 |
| Ammonium sulphate | 0.50 | 0.50 |
| Sodium sulphite | 0.5 | 0.5 |
| Ammonia, 25% | 7.0 | 7.0 |
| Preservative | q.s. | q.s. |
| Perfume | q.s. | q.s. |
| Water | ad 100 | ad 100 |
| Oxidation dye mixture: | | |
| 2,5,6-Triamino-4-hydroxypyrimidine sulphate | 0.01 | 0.01 |
| 2,5-Diaminotoluene sulphate | 0.55 | 0.55 |
| 4-Chlororesorcinol | 0.17 | 0.17 |
| 3-Aminophenol | 0.03 | 0.03 |

1.-15. (canceled)

16. A cosmetic composition comprising one or more aqueous polyurethane dispersions and a keratin material colorant component.

17. The cosmetic composition according to claim 16, wherein the keratin material is human or animal hair.

18. The cosmetic composition according to claim 16, wherein the one or more aqueous polyurethane dispersions comprise the reaction product of
A1) one or more organic polyisocyanates,
A2) one or more polymeric polyols having a number-average molecular weight of 400 to 8000 g/mol as determined by gel permeation chromatography against polystyrene standard in tetrahydrofuran at 23° C. and having 1.5 to 6 OH functionalities,
A3) optionally one or more hydroxy-functional compounds having a molecular weight of 32 to less than 400 g/mol,
A4) optionally one or more isocyanate-reactive hydrophilizing agents, and
B1) optionally one or more amino-functional compounds.

19. The cosmetic composition according to claim 18, wherein the one or more isocyanate-reactive hydrophilizing agents are selected from the group consisting of anionic hydrophilizing agents, potentially anionic hydrophilizing agents, nonionic hydrophilizing agents, and mixtures thereof.

20. The cosmetic composition according to claim 18, wherein the one or more amino-functional compounds is used in the reaction and has a molecular weight of 32 to 400 g/mol.

21. The cosmetic composition according to claim 18, wherein the one or more organic polyisocyanates comprises polyisocyanates or polyisocyanate mixtures with exclusively aliphatically and/or cycloaliphatically bonded isocyanate groups and having an average NCO functionality of the mixture of from 2 to 4.

22. The cosmetic composition according to claim 18, wherein the one or more polymeric polyols exclusively comprises a mixture of at least two polytetramethylene glycol polyethers with different number-average molecular weights.

23. The cosmetic composition according to claim 22, wherein the one or more polymeric polyols exclusively comprises two polytetramethylene glycol polyethers, and the polytetramethylene glycol polyether with the lower molecular weight is present in an amount of 3 to 80% by weight, based on the total amount of polytetramethylene glycol polyethers.

24. The cosmetic composition according to claim 23, wherein the number-average molecular weight Mₙ of the polytetramethylene glycol polyether with the lower molecular weight is in the range of from 650 to 1400 g/mol and the number-average molecular weight Mₙ of the polytetramethylene glycol polyether with the higher molecular weight is in the range of from 1500 to 4000 g/mol.

25. The cosmetic composition according to claim 16, wherein the aqueous polyurethane dispersion is present in an amount of 0.1 to 20% by weight, based on the total composition.

26. The cosmetic composition according to claim 16, wherein the keratin material colorant component is selected from the group consisting of one or more direct dyes, at least one oxidation dye precursor, and mixtures thereof.

27. The cosmetic composition according to claim 26, wherein the one or more direct dyes is present in an amount of up to 20% by weight of the total composition.

28. The cosmetic composition according to claim 26, wherein the at least one oxidation dye precursor constitutes up to 5% by weight of the total composition.

29. The cosmetic composition according to claim 27, wherein the at least one oxidation dye precursor constitutes up to 5% by weight of the total composition.

30. The cosmetic composition according to claim 16, wherein the cosmetic composition is selected from the group consisting of a lotion, a cream, and a foam.

31. A pump-spray package, an aerosol package or a foam dispenser, comprising the cosmetic composition according to claim 16.

32. A process comprising:
providing a keratin-containing material, and
applying the composition of claim 16 to the keratin-containing material.

33. The process according to claim 32 wherein the keratin-containing material is human or animal hair.
